(19) United States
(12) Patent Application Publication
(10) Pub. No.: US 2004/0092443 A1
(43) Pub. Date: May 13, 2004

(54) Long-acting exendins and exendin agonists

(75) Inventors: Matityahu Fridkin, Rehovot (IL); Yoram Shechter, Rehovot (IL); Haim Tsubery, El-Ad (IL)

Correspondence Address: BROWDY AND NEIMARK, P.L.L.C., 624 NINTH STREET, NW, SUITE 300, WASHINGTON, DC, 20001-5303, US

(73) Assignee: YEDA Research and Development Co., Ltd, Rehovot (IL)

(21) Appl. No.: 10/408,262

(22) Filed: Apr. 8, 2003

Related U.S. Application Data
(63) Continuation-in-part of application No. 10/336,839, filed on Jan. 6, 2003.
Application No. 10/336,839 is a continuation of application No. 09/242,026, filed on Feb. 5, 1999, now Pat. No. 6,504,005.
Application No. 09/242,026 is a 371 of international application No. PCT/IL97/00265, filed on Aug. 5, 1997.

(30) Foreign Application Priority Data
Aug. 6, 1996 (IL) 119029

(51) Int. Cl. A61K 38/28; C07K 14/47

(52) U.S. Cl. 514/12; 530/409

(57) ABSTRACT

Long-acting exendin or exendin agonist derivatives of the formula (X)ₙ-Z are provided, wherein X is a radical 9-fluorenylmethoxycarbonyl (Fmoc) or 2-sulfo-9-fluorenyl-methoxycarbonyl (FMS), Z is the residue of an exendin or exendin agonist linked to the radical X through an amino or hydroxyl group, and n is 1 to 3. The exendin is exendin-3 or exendin-4. The derivatives are useful for prevention or treatment of conditions, diseases or disorders that can be treated by an exendin, for example for prevention of hyperglycemia and for treatment of diabetes mellitus, e.g. non-insulin dependent diabetes mellitus, insulin-dependent diabetes mellitus, and gestational diabetes mellitus.

### CROSS-REFERENCE TO RELATED APPLICATIONS

The present application is a continuation-in-part application of copending U.S. application Ser. No. 10/336,839, filed Jan. 6, 2003, which is a continuation of U.S. application Ser. No. 09/242,026, filed Feb. 5, 1999, now U.S. Pat. No. 6,504,005, granted Jan. 7, 2003. The U.S. application Ser. No. 09/242,026 claimed priority benefit under 35 U.S.C. 371 of PCT/IL97/00265, filed Aug. 5, 1997, and claimed priority benefit under 35 U.S.C. 119 of Israeli Patent Application No. 119029, filed Aug. 6, 1996. The contents of each of applications No. 10/336,839 and No. 09/242,026 is hereby incorporated herein by reference in its entirety.

### FIELD OF THE INVENTION

The present invention relates to novel long-acting derivatives of exendins and exendin agonists that following administration are capable of undergoing spontaneous chemical transformation in the body from an inactive into a bioactive form, and particularly to derivatives of exendins and exendin agonists bearing functional groups sensitive to mild basic conditions, and to pharmaceutical compositions comprising them for treatment of diabetes mellitus and the prevention of hyperglycemia.

Abbreviations: Fmoc: 9-fluorenylmethoxycarbonyl; Fmoc-OSu: 9-fluorenylmethoxycarbonyl N-hydroxysuccinimide ester; FMS: 2-sulfo-9-fluorenylmethoxycarbonyl; FMS-OSu: 2-sulfo-9-fluorenylmethoxycarbonyl N-hydroxysuccinimide ester; GDM: gestational diabetes mellitus; IDDM: insulin-dependent diabetes mellitus; NIDDM: non-insulin dependent diabetes mellitus.

### BACKGROUND OF THE INVENTION

Glucagon-like peptide-1 (7-37) (7-36)-amide (GLP-1) is one of the intestinal peptide hormones that are released into the circulatory system after food intake. It augments the postprandial release of insulin, when nutrients (especially carbohydrates) are absorbed and their level postprandially elevated. GLP-1 associates with GLP-1 receptor sites located on pancreatic β-cells and elevates endogenous cAMP levels in a dose-dependent manner (Göke et al., 1993). In isolated rat islets in the presence of above normoglycemic glucose levels, GLP-1 stimulates the release of insulin. Infusion of GLP-I to normal subjects, or to non-insulin dependent diabetes mellitus (NIDDM) patients, enhanced secretion of insulin and lowered the meal-related increase in blood glucose concentration. It also decreased the release of glucagon and somatostatin. In addition to glucose-dependent insulinotropic action, GLP-1 was also documented to restore islet glucose sensitivity, known to be glucagonostatic in nature, to slow gastric emptying, and possibly to play a physiological role in appetite control. A therapeutic potential for GLP-1 in NIDDM patients was suggested almost a decade ago, owing to the profound efficacy of this insulinotropic peptide to stimulate the secretion of insulin when glucose levels are elevated and to cease doing so upon return to normoglycemia (Gutnick et al., 1992).

Exendin-4, a 39-amino acid peptide isolated from the salivary secretions of the venomous lizard Gila monster (Heloderma suspectum), shows 53% amino acid identity to GLP-I and acts as a GLP-1 agonist (Göke et al., 1993; Eng et al., 1992). It associates with GLP-1 receptors located on pancreatic β-cells with 2.5 times higher affinity (Kd=0.136 nM) than GLP-1. In isolated rat islets, in the presence of 10 mM glucose, exendin-4 enhances secretion of insulin in a dose-dependent fashion (Göke et al., 1993). Studies in type II diabetic rodents revealed that exendin-4 is 5530-fold more potent than GLP-i in lowering blood glucose levels. Also, the duration of glucose-lowering action after a single administration of exendin-4 is significantly longer compared to GLP-1 (Young et al., 1999).

Exendin-3 is a peptide isolated from the salivary secretions of the venomous lizard Heloderma horridum. Both exendin-3 and exendin-4 stimulate cAMP production in pancreatic acinar cells. U.S. Pat. No. 5,424,286 (John Eng, 1995) discloses the use of exendin-3, exendin-4 or exendin-4(1-31) fragment, as insulinotropic agents, namely as agents capable of stimulating or causing the stimulation of the synthesis or expression of insulin, and therefore useful for the treatment of diabetes mellitus, and the prevention of hyperglycemia.

Exendin agonist compounds have been described for regulating gastrointestinal mobility, for reduction of food intake, for glucagons suppression, and for treatment of gestational diabetes mellitus (GDM). Both the exendins and the exendin agonists do not cross the placenta, and yet have a prolonged effect on the blood glucose, rendering them ideal agents for treatment of GDM (U.S. Pat. No. 6, 506,724).

In the parent U.S. application Ser. No. 10/336,839 and U.S. application Ser. No. 09/242,026, now U.S. Pat. No. 6,504,005, a novel conceptual approach for generation of long-acting drugs is disclosed by derivatizing a drug having at least one free amino, carboxyl, hydroxyl and/or mercapto groups with a moiety that is highly sensitive to bases and is removable under mild basic conditions. The prodrug obtained is inactive but undergoes transformation into the active drug under physiological conditions in the body. Examples of said moieties are the radicals 9-fluorenylmethoxycarbonyl (Fmoc) and 2-sulfo-9-fluorenylmethoxycarbonyl (FMS). According to this concept, Fmoc and FMS derivatives of peptidic drugs such as insulin (Gershonov et al., 1999; 2000) and human growth hormone, as well as of non-peptidic drugs such as propanolol, cephalexin and piperacillin (U.S. Pat. No. 6,504,005 and WO 98/05361), of cytokines (WO 02/36067; Shechter et al., 2001) and of enkephalin, doxorubicin, amphotericin B, gentamicin and gonadotropin releasing hormone (GnRH) (WO 02/7859), have been described.

Citation of any document herein is not intended as an admission that such document is pertinent prior art, or considered material to the patentability of any claim of the present application. Any statement as to content or a date of any document is based on the information available to applicants at the time of filing and does not constitute an admission as to the correctness of such a statement.

### SUMMARY OF THE INVENTION

The present invention relates to an exendin or exendin agonist derivative of the formula:

(X)ₙ-Z

wherein X is a radical 9-fluorenylmethoxycarbonyl (Fmoc) or 2-sulfo-9-fluorenylmethoxycarbonyl (FMS), Z is the residue of an exendin or exendin agonist linked to the radical X through an amino or hydroxyl group, and n is 1 to 3.

The exendin may be exendin-3 or exendin-4 and the agonist may be an exendin-3 agonist or exendin-4 agonist.

The present invention further relates to pharmaceutical compositions comprising a pharmaceutically acceptable carrier and at least one derivatives of an exendin or exendin agonist according to the invention.

The present invention still further relates to methods for prevention and/or treatment of conditions, diseases or disorders that can be treated with an exendin or exendin agonist, particularly prevention of hyperglycemia and treatment of diabetes mellitus, by administering to an individual in need an effective amount of an exendin or exendin agonist derivative of the invention.

### BRIEF DESCRIPTION OF THE DRAWINGS

FIG. 1 is a graph showing the rate of FMS-hydrolysis from (FMS)₃-exendin-4 at pH 8.5, 37° C. A solution of (FMS)₃-exendin-4 (0.07 mM in 0.1 M NaHCO₃, pH 8.5) was incubated at 37° C. At the indicated time points, aliquots (0.4 ml) were withdrawn and free amino side chains were quantitated with 2,4,6-trinitrobenzenesulfonic acid (TNBS).

FIG. 2 is a graph showing glucose-lowering pattern of native exendin-4 administered subcutaneously to CD1 mice, at 5 indicated concentrations (exendin-4 was dissolved in 0.1 ml PBS buffer, 0.1% BSA). At the indicated time points, circulating glucose levels were determined. Each experimental group consisted of five mice. t_{1/2} values are indicated for each concentration of exendin-4. Data are presented as means±SE.

FIG. 3 is a graph showing the intrinsic glucose-lowering potency of (FMS)₃-exendin-4 prior to FMS hydrolysis compared to native exendin-4. The indicated concentrations of native-exendin-4 and of (FMS)₃-exendin-4 were administered subcutaneously to CD 1 mice (n=5 per each group). Circulating glucose levels were determined one hour after administration. Results are expressed as % of maximal glucose-lowering capacity, where 100% is the effect manifested by administering 1 μg (250 picomoles) of native exendin-4 per mouse.

FIG. 4 is a graph showing glucose-lowering patterns of (FMS)₃-exendin-4 following subcutaneous administration to CD1 mice, at 1, 10 and 100 μg/mouse (n=5 per each group). Circulating glucose levels were monitored at the indicated time points after administration. t_{1/2} values are indicated for each concentration of exendin-4. Data are presented as means±SE.

FIG. 5 is a graph showing glucose-lowering patterns of native exendin-4 and (FMS)₃-exendin-4, following a single subcutaneous administration to db/db mice. Three groups of db/db mice (n=4 per group) were administered subcutaneously either with saline, native exendin-4 (10 μg/mouse), or (FMS)₃-exendin-4 (10 μg/mouse). Circulating glucose levels were then monitored. Results are expressed as percent decrease in plasma glucose concentration in the exendin-4- and the (FMS)₃-exendin-4-treated groups relative to saline-treated group, measured at the same time point during the day.

### DETAILED DESCRIPTION OF THE INVENTION

The present invention provides an exendin or exendin agonist derivative of the formula (X)ₙ-Z, wherein X is a Fmoc or FMS radical, Z is the residue of an exendin or exendin agonist linked to the Fmoc or FMS radical X through an amino or hydroxyl group, and n is 1 to 3.

In one embodiment, the derivative is a Fmoc or FMS derivative of an exendin peptide, either exendin-4 [SEQ. ID. NO: 1] or exendin-3 [SEQ. ID. NO:2] of the sequences below:

Exendin-4:

[SEQ. ID. NO:1]

HGEGTFTSDL SKQMEEEAVR LFIEWLKNGG PSSGAPPPS-NH₂
Exendin-3

[SEQ. ID. NO:2]

HSDGTFITSDL SKQMEEEAVR LFIEWLKNGG PSSGAPPPS-NH₂

In one preferred embodiment, the exendin is exendin-4 and the exendin-4 derivative is selected from the group consisting of the derivatives herein designated Fmoc-exendin-4, (Fmoc)₂-exendin-4, (Fmoc)₃-exendin-4, FMS-exendin-4, (FMS)₂-exendin-4 and, most preferably, (FMS)₃-exendin-4.

In another embodiment, the exendin is exendin-3 and the exendin-3 derivative is selected from the group consisting of the derivatives herein designated Fmoc-exendin-3, (Fmoc)₂-exendin-3, (Fmoc)₃-exendin-3, FMS-exendin-3, (FMS)₂-exendin-3 and (FMS)₃-exendin-3.

In a further embodiment, the derivative is a Fmoc or FMS derivative of an exendin agonist derivative wherein said exendin agonist is an exendin-3 or exendin-4 agonist. As used herein, an “exendin agonist” is a compound, preferably a peptide, that mimics the activities of exendin-3 or exendin-4 by binding to the receptor(s) at which exendin-3 or exendin-4 exerts its actions which are beneficial as insulinotropic and in the treatment of diabetes mellitus or by mimicking the effects of exendin on increasing urine flow, increasing urinary sodium excretion and/or decreasing urinary potassium concentration, by binding to the receptor(s) where exendins cause these effects.

Exendin agonists have been described in U.S. Pat. No. 5,424,286, U.S. Pat. No. 6,506,724, WO 99/07404, WO 99/25727, WO 99/25728, and WO 99/40788, each and all of these patents and patent applications being hereby incorporated herein by reference as if fully disclosed herein.

Examples of exendin agonists that can be used to prepare the long-acting exendin derivatives according to the invention include, but are not limited to, exendin-4 agonists selected from the group consisting of:

(i) exendin-4 and amidated exendin-4, in which sequences one or more amino acid residues have been replaced by different amino acid residues

(ii) truncated exendin-4 and truncated forms that are amidated; and

(iii) truncated exendin-4 and truncated forms that are amidated, in which sequences one or more amino acid residues have been replaced by different amino acid residues.

The following insulinotropic exendin-4 fragments and analogues of the SEQ ID NOs: 3-10 can be used to prepare the long-acting derivatives of the invention:

| | | | |
|---|---|---|---|
| | exendin-4 (1-31) | HGEGTFTSDLSKQMEEEAVRLFIEWLKNGGP; | [SEQ ID No:3] |
| Y³¹ | exendin-4 (1-31) | HGEGTFTSKLSKQMEEEAVRLFIEWLKNGGY; | [SEQ ID No:4] |
| | exendin-4 (1-30) | HGEGTFTSDLSKQMEEEAVRLFIEWLKNGG; | [SEQ ID No:5] |
| | exendin-4 (1-30) amide | HGEGTFTSDLSKQMEEEAVRLFIEWLKNGG-NH₂; | [SEQ ID No:6] |
| | exendin-4 (1-28) amide | HGEGTFTSDLSKQMEEEAVRLFIEWLKN-NH₂; | [SEQ ID No:7] |
| L¹⁴, F²⁵ | exendin-4 amide | HGEGTFTSDLSKQLEEEAVRLFIEFLKNGGPSSGAPPPS-NH₂; | [SEQ ID No:8] |
| L¹⁴, F²⁵ | exendin-4 (1-28) amide | HGEGTFTSDLSKQLEEEAVRLFIEFLKN-NH₂; and | [SEQ ID No:9] |
| L¹⁴, A²², F²⁵ | exendin-4 (1-28) amide | HGEGTFTSDLSKQLEEEAVRLAIEFLKN-NH₂. | [SEQ ID No:10] |

Other examples of exendin agonists that can be used according to the invention may be chosen from those agonists known to mimic the insulinotropic effect exhibited by exendin or agonists that will be found in the future.

Exendin-4 and exendin-3 and agonists thereof have been found to lower excess levels of blood glucose (U.S. Pat. No. 6,528,486) and to be potentially useful for treatment of diabetes mellitus type I (IDDM) and type II (NIDDM) (WO 99/07404, WO 99/25727, WO 99/25728), and gestational diabetes mellitus (U.S. Pat. No. 6,506,724). They have also been disclosed as useful in methods for increasing urine flow, increasing urinary sodium excretion and decreasing urinary potassium concentration, and thus for treating conditions or disorders associated with toxic hypervolemia, such as renal failure, congestive heart failure, nephrotic syndrome, cirrhosis, pulmonary edema, and hypertension, for inducing an inotropic response and thus being useful for treating conditions or disorders that can be alleviated by an increase in cardiac contractility such as congestive heart failure (WO 99/40788). The compounds have in addition been disclosed as capable of regulating gastric motility and delaying and/or slowing gastric emptying (WO 99/07404).

The exendin and exendin agonists derivatives of the invention will exert any and all activities exhibited by the parent non-modified molecule but with a prolonged action. The derivative is administered as a prodrug being essentially non-active biologically but being capable of spontaneous and slow conversion to the original active drug molecule in its bioactive form under physiological conditions in the body, following administration.

Thus, in another aspect, the present invention relates to a pharmaceutical composition comprising an exendin or exendin agonist derivative of the invention, and a pharmaceutically acceptable carrier. These compositions are for use for any of the uses known or to be discovered in the future for exendin and exendin agonists, for example, for prevention of hyperglycemia and for treatment of diabetes mellitus of any type, e.g. insulin-dependent diabetes mellitus (type I or IDDM), non-insulin dependent diabetes mellitus (type II or NIDDM), or gestational diabetes mellitus (GDM).

The compositions useful in the invention may be presented in any suitable route of administration to humans such as formulations for parenteral, including intravenous, intramuscular and subcutaneous, or for intranasal or oral administration. Suitable pharmaceutically acceptable carriers and excipients can be added by conventional methods known to those skilled in the art, for example as described in Remington: The Science and Practice of Pharmacy, A. R. Gennaro, ed., 20th edition, 2000.

In another aspect, the present invention relates to a method for prevention or treatment of a condition, disease or disorder that can be prevented or treated with an exendin or exendin agonist, which comprises administering to an individual in need an effective amount of an exendin or exendin agonist derivative of the invention.

In one embodiment, the present invention relates to a method for prevention of hyperglycemia which comprises administering to an individual in need an effective insulinotropic amount of an exendin or exendin agonist derivative of the invention.

In another embodiment, the present invention provides a method for treatment of diabetes mellitus which comprises administering to an individual in need an effective amount of an exendin or exendin agonist derivative of the invention. The diabetes mellitus may be non-insulin dependent diabetes mellitus, insulin-dependent diabetes mellitus, or gestational diabetes mellitus.

The exendin and exendin derivatives of the invention may be obtained, for example, by reacting the exendin or exendin agonist with excess of 9-fluorenylmethoxycarbonyl N-hydroxy-succinimide ester (Fmoc-OSu) or 2-sulfo-9-fluorenylmethoxycarbonyl N-hydroxy-succinimide ester (FMS-Osu), reagents which are very specific for amino groups, or with 9-fluorenylmethoxycarbonyl chloride (Fmoc-Cl), that reacts with, and covalently attaches to, amino and hydroxyl radicals.

In a most preferred embodiment of the present invention, three molecules of FMS were covalently linked to three amino functions of exendin-4 of SEQ. ID. NO: 1: one FMS radical is linked to the α-amino group of the N-terminal residue of exendin-4 and the other two FMS radicals are linked to the ε-amino groups of two lysine (K) residues at positions 12 and 27 of exendin-4. The (FMS)₃-exendin-4 was prepared as described above by reacting one equivalent of exendin-4 with 15 equivalents of solid FMS-OSu.

The prodrug of the invention (FM S)₃-exendin-4 has about 0.1% the glucose-lowering potency of the native peptide exendin-4. Upon incubation of (FMS)₃-exendin-4 at 37° C. under physiological conditions, the derivative undergoes FMS hydrolysis in a slow, nearly linear and spontaneous fashion, generating the non-modified exendin-4 molecule with a t_{1/2} value of 18±2 hrs.

To evaluate the efficacy of the (FMS)₃-exendin-4 derivative in lowering blood glucose level, the glucose-lowering patterns of the native hormone exendin-4 and of the derivative were examined in control and diabetic mice. A single subcutaneous administration of (FMS)₃-exendin-4 facilitated a dramatically prolonged, dose-dependent glucose-lowering pattern. The typical pharmacokinetic profile of (FMS)₃-exendin-4 after subcutaneous administration shows a modest glucose lowering effect within the first hour of administration, blood glucose level then falls reaching its lowest value at two to four hours after administration. At the highest dose of (FMS)₃-exendin-4 applied, stable, low circulating glucose is maintained over many hours, exceeding two days (t_{1/2} value=44±3 hrs).

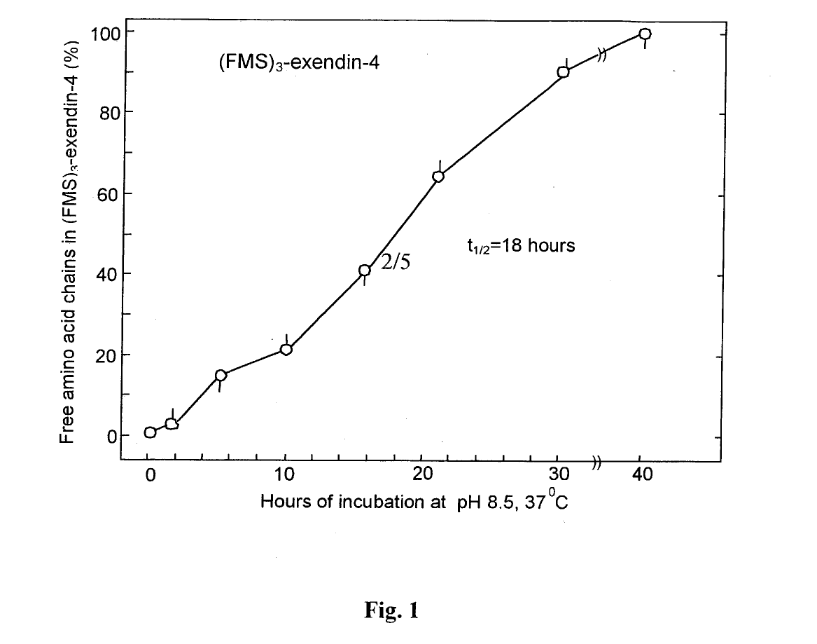

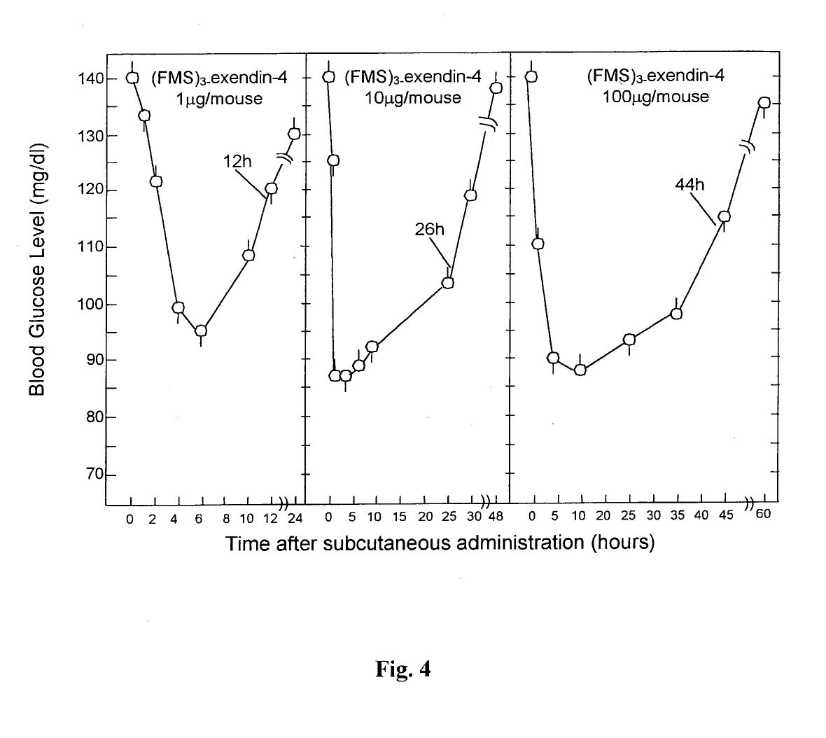

The unique feature of exendin-4 (and of (FMS)₃-exendin-4 as well) to remain in a nonfunctioning, “silent” state, below a certain threshold glucose level is demonstrated by the present invention. For example, healthy, non-diabetic mice are characterized by higher fasting glucose levels (namely, 140±10 mg/dl, 7.7710.05 mM) compared to rats or humans (90±5 mg/dl, 5.0±0.03 mM). As shown pursuant to the invention, the minimal concentration of plasma glucose at any dose applied, and at any time point after exendin-4 administration, never fell below 87.0±3 mg/dl (4.8310.02 mM glucose), namely, the normal fasting glucose levels in rats or humans. In agreement with this, subcutaneous administration of excess exendin-4 to rats did not significantly reduce plasma glucose levels in the animals (not shown). Thus, with respect to circulating glucose levels, two boundary conditions can be discriminated: a low boundary of 4.83 mM glucose, below which exendin-4 is completely incapable of lowering plasma glucose any further, and an upper boundary of 7.77 mM glucose, a value which appears sufficient for exendin-4 to lower circulating glucose level to its maximal capacity (FIGS. 1 and 4).

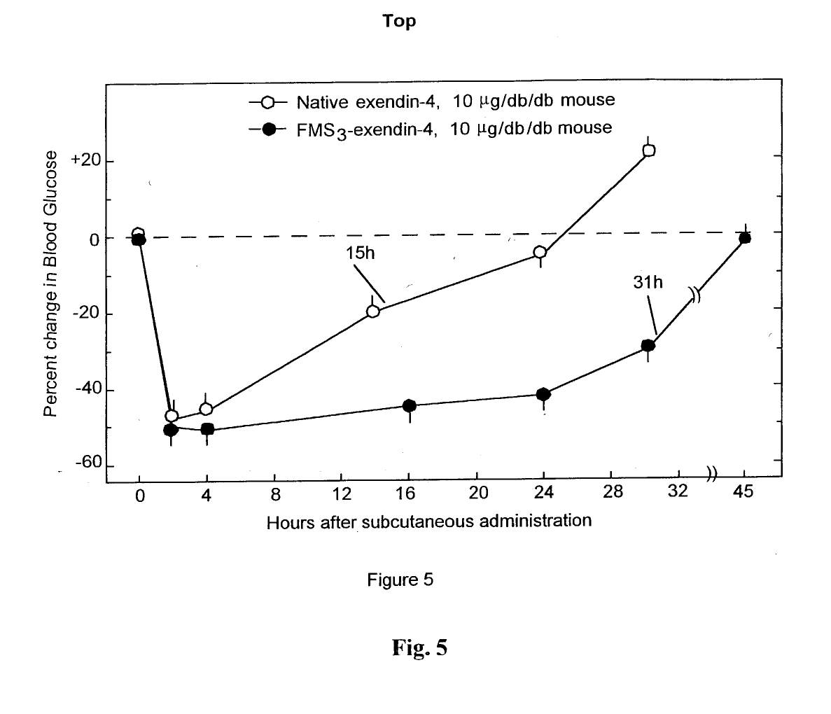

When comparing the glucose-lowering patterns in healthy and diabetic mice following administration of exendin-4 and the of (FMS)₃-exendin-4 derivative, it was found that the magnitude of the glucose-lowering effect was not significantly different in healthy and diabetic mice, being ˜39% and ˜48%, respectively (FIGS. 1 and 5). Also, the glucose-lowering pharmacokinetic profiles were nearly identical (FIG. 4 versus FIG. 5), indicating that clearance and/or inactivation of exendin-4 in vivo were similar in both experimental groups. A significant difference, however, was found in the ED₅₀ values. Those were 0.2±0.01 and 1.0±0.1 μg of exendin-4 per kg of db/db and CD1 mice, respectively (not shown). Thus, the diabetic mouse is about five fold more sensitive to exendin-4 in vivo.

The invention will now be illustrated by the following non-limiting Examples.

### EXAMPLES

Materials and Methods

(i) Materials. Exendin-4 (SEQ ID NO:1) was synthesized by the solid phase peptide synthesis method using a multiple-peptide synthesizer (AMS 422, ABIMED Analysen-Technik Gmbh, Langenfeld, Germany). Fmoc-OSu was obtained from Novabiochem (Läufelfingen, Switzerland). All other materials used were of analytical grade.

(ii) Animals. Genetically diabetic mice db/db mice (strain name B6.Cg-m+/+Lepr^{db}, mean weight 48±3), were obtained from Jackson Laboratories (Bar Harbor, Me., USA). Mice were kept at 22±1° C., 55±10% relative humidity, 14:10 hours light-dark cycle. Experiments were performed three weeks after db/db mice transportation.

(iii) Reverse-phase HPLC was performed with a Spectra-Physics SP8800 liquid chromatography system (Spectro-Physics, San Jose, Calif.) equipped with an Applied Biosystem 757 variable wavelength absorbance detector. The column effluents were monitored by UV absorbance at 220 nm and chromatograms were recorded on a chrom-Jet Integrator (Thermo-Separation, Riviera Beach, Fla.). HPLC prepacked columns used included LiChroCART 250-10 mm containing LiChrosorb RP-18 (7 μm) and LiChrospher 100 RP-18 (5 μm), 250-4 mm (Merck, Rahway, N.J.). Linear gradients were used between solution A (0.1% TFA in H₂O) and solution B (0.1% TFA in acetonitrile-H₂O, 75:25). For analytical HPLC procedures, a linear gradient between 30 and 100% of solution B was run for 50 min at a flow rate of 0.8 mL/min.

(iv) Mass spectroscopy: Mass analyses were carried out by electrospray ionization mass spectra (ESMS) technique, or with matrix-assisted laser-desorption/ionization-time-of-flight (MALDI-TOF) mass spectroscopy (Micromass UK Ltd.). The polypeptides were deposited on a metal target as cocrystals with sinaptic acid, and the mass spectrum was determined in the positive ion mode.

(v) Blood glucose levels were measured with a glucose analyzer (Beckman Instrument, Fullerton, Calif.) by the glucose oxidase method. Blood samples for the analysis of blood glucose were taken from the tail veins. The level of glucose in normal healthy CD1-mice was 140±7 mg/dl (7.77 mM). Each experimental group consisted of 5 mice. Data are presented as means±SE.

### Example 1

### Synthesis of FMS-OSu (2-sulfo-9-fluorenylmethoxycarbonyl N-hydroxy-succinimide Ester)

FMS-OSu was prepared by sulfonation of Fmoc-OSu essentially as follows: Fmoc-OSu (337.4 mg, 1 mmol) was dissolved in 4 ml of dichloromethane and cooled to 0° C. A solution of ClSO₃H (60 μl, 0.9 mmol) in 2 ml of dichloromethane was added with constant stirring and cooling over a period of 15 nun. The yellow-turning solution was allowed to warm to room temperature, and a white precipitate was formed within 1 h. At 2 h, cyclohexane (4 ml) was added to dissolve the unreacted Fmoc-OSu. The suspension was centrifuged and washed four times with 6 ml of 1:1 vol/vol) cyclohexane/dichloromethane. The white solid thus formed was dried under P₂O₅ in vacuo for 24 h and had the following characteristics: yield—290 mg (86%); mp 140-146° C.; TLC (1-butanol/acetic acid/water, 8:1:1) Rf 0.31, and mass spectrum (ES⁻) m/z 416 (100%, M-1). FMS moieties, either free or covalently bound to proteins, absorb at the UV range with molar extinction coefficients ε₂₈₀=21,200 and ε₃₀₁=10,300.

### Example 2

### Synthesis of (FMS)₃-exendin-4

(FMS)₃-exendin-4 was prepared by dissolving 4.2 mg exendin-4 (1 μmole) in 1.0 ml of 0.1M sodium bicarbonate and the solution cooled to 0° C. Solid FMS-OSu obtained in Example 1 (6.3 mg, 15 μmoles, 15 molar excess) was added to the stirred peptide solution in several aliquots over a period of 40 min. The reaction mixture was then dialyzed against H₂O at 7° C. with several changes over a period of 2 days. A fraction of the (FMS)₃-exendin-4 thus obtained (1 mg) was further purified by analytical HPLC-procedure to yield 0.8 mg of pure (FMS)₃-exendin-4. Native exendin and (FMS)₃-exendin-4 absorb at the UV region with molar extinction coefficient ε₂₈₀=5,600±200 and 68,900±400, respectively.

### Example 3

### Chemical Characterization of (FMS)₃-exendin-4

Table 1 summarizes the main chemical features of HPLC-purified (FMS)₃-exendin-4 prepared in Example 2. The compound contains three mol of FMS per mol peptide as deduced by its adsorption spectrum at 280 nm (ε₂₈₀=68,900±400), a value corresponding to the absorption of 3 mol of FMS plus that of the native peptide. Mass spectrum analyses (MALDI-TOF) yielded a mass of 4184.77 daltons for native exendin-4 and a mass of 5092 daltons for (FMS)₃-exendin-4 (calculated MW=5090 daltons). The derivative is soluble in aqueous solution (pH 7.0 and above). It migrates as a doublet peak (protonized and partially non-protonized sulfo functions) on HPLC, with a retention time values of 29.7 and 30.1 min.

TABLE 1
Chemical Features of (FMS)₃-exendin-4

| Characteristic | (FMS)₃-exendin-4 |
|---|---|
| Amino acid composition | identical to exendin-4 |
| mol FMS/rnol exendin-4^{a} | 3.0 ± 0.1 |
| Solubility in aqueous buffer, pH 7.4 | >3.0 mg/ml |
| Absorbance at 280 nm^{b} | ε₂₈₀ = 68,900 ± 400 |
| Retention time (analytical HPLC)^{c} | Doublet, Rₜ = 29.7 and |
| | 30.1 minutes |
| Mass spectra^{d} | Calculated: 5090 daltons; |
| | Found: 5092 daltons |

(a) Determined by UV spectroscopy, measuring the absorbance at 280 nm. Derivative concentration was determined by acid hydrolysis of a 20 μl aliquot, followed by amino acid analysis, calculated according to aspartic acid (2 residues), alanine (2 residues), and isoleucine (1 residue).

(b) Native exendin-4 absorbs at 280 nm with ε₂₈₀=5,600±200.

(c) Reverse-phase HPLC was performed with a Spectra-Physics SP8800 liquid chromatography system, under the experimental conditions described in detail in Gershonov et al., 2000. Native-exendin-4 was eluted under identical analytical HPLC procedure with retention time=27.7±0.2 min.

(d) Mass spectra were determined by the MALDI-TOF mass spectroscopy. A mass of 4185 daltons has been calculated and found for native-exendin-4.

### Example 4

### Rate of hydrolysis of (FMS)₃-exendin-4

(FMS)₃-exendin-4 is 2,4,6-trinitrobenzenesulfonic acid (TNBS) negative as all three amino functions of exendin-4 are derivatized. In the experiments summarized in FIG. 1, (FMS)₃-exendin-4 was incubated in 0.1M NaHCO₃ (pH 8.5) at 37° C. and aliquots withdrawn at different time points were analyzed for the appearance of free amino groups with TNBS. At pH 8.5, the rate of FMS hydrolysis from the FMS-peptide and protein conjugates (e.g. insulin or IFN-α) was nearly the same as that found in vivo or in human serum in vitro. Therefore, this pH was selected to effect FMS₃-exendin hydrolysis in subsequent experiments. FMS moieties of (FMS)₃-exendin-4 were hydrolyzed in slow and homogeneous fashion with a t_{1/2} value of 18±2 hrs. Hydrolysis was complete after 40 hrs of incubation (FIG. 1).

### Example 5

### Glucose-Lowering Action of Native Exendin-4 in Non-Diabetic Mice

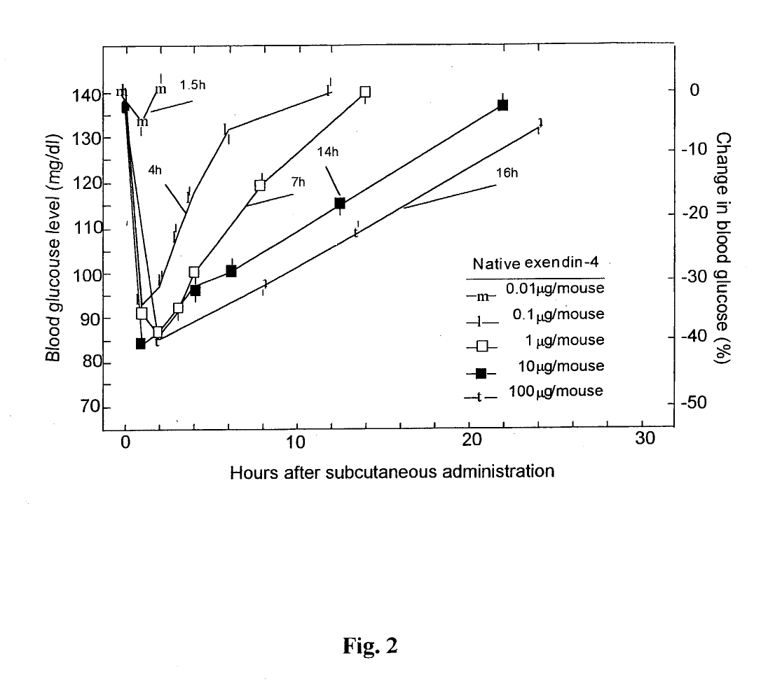

The glucose-lowering pattern of the native exendin-4 is shown in FIG. 2. In all cases, exendin-4 was administered subcutaneously to CD1 mice (0.1 ml per mouse) in phosphate-buffered saline (PBS) buffer (pH 7.4) containing 0.1% bovine serum albumin (BSA). Exendin-4 at 0.01 μg/mouse had little effect on reducing blood glucose levels (FIG. 2). However, at doses of 0.1 μg/mouse or higher, circulating glucose levels were significantly reduced. The lowest glucose level was obtained within 1 h after administration and amounted to a decrease of 3913% of initial value (a decline from 14017 to 86±4 mg/dl). No further decrease in circulating glucose level was obtained at any dose applied or at any time point after administration. With exendin-4 doses of 0.1, 1.0, 10.0 and 100 μg/mouse, circulating glucose levels returned to normoglycemic values with t_{1/2} values of 4±0.3, 7.0±0.4, 14±2 and 16±3 h, respectively.

### Example 6

### Intrinsic Glucose-Lowering Potency of (FMS)₃-exendin-4 in Healthy Mice

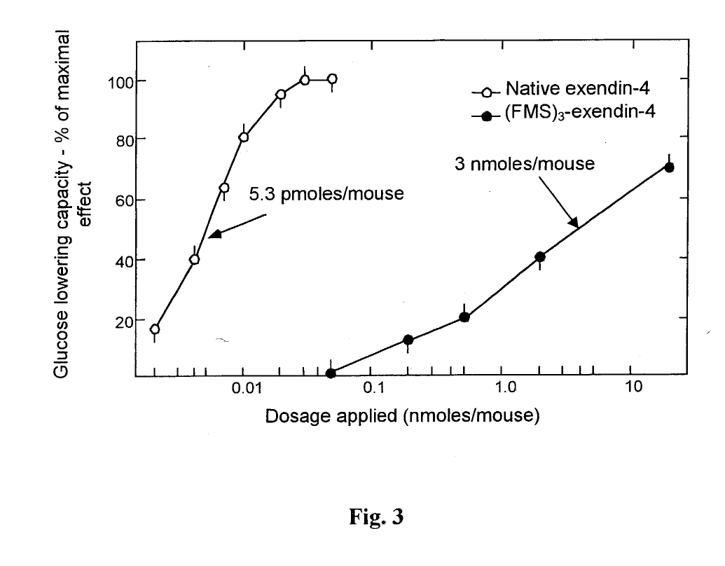

To determine the glucose-lowering potency of (FMS)₃-exendin-4 prior to FMS hydrolysis, increasing doses of the native peptide and of (FMS)₃-exendin-4 were subcutaneously administered to non-diabetic CD1 mice and the relative glucose-lowering efficacy (% of maximal) was assessed 1 h after administration. At this early time point, (FMS)₃-exendin-4 undergoes negligible FMS-hydrolysis and reactivation, either in vitro or in vivo. As shown in FIG. 3, the native exendin-4 and (FMS)₃-exendin-4 induced their half maximal glucose lowering potencies at concentrations of 5.3±0.4 picomoles and 3+0.3 nmoles/mouse, respectively. Thus, prior to FMS-hydrolysis, (FMS)₃-exendin-4 possesses 0.1% the glucose-lowering potency of the native exendin-4.

### Example 7

### (FMS)₃-exendin-4 Facilitates Prolonged Glucose-Lowering Action in Healthy Mice

The pharmacokinetic glucose lowering profiles of (FMS)₃-exendin-4 in CD1 mice are shown in FIG. 4. The experiment included three doses of (FMS)₃-exendin-4, namely, 1.0, 10.0 and 100 μg/mouse administered subcutaneously. (FMS)₃-exendin-4 facilitated a prolonged glucose-lowering action, with the lowest glucose concentrations reached 2-6 hours after administration. Stable, low circulating glucose concentrations were then maintained over many hours. The return to initial glucose levels occurred with t_{1/2} values of 12±0.7, 26±2 and 44±3 hrs following administration of 1, 10 and 100 μg/mouse of (FMS)₃-exendin-4, respectively.

### Example 8

### Subcutaneously Administered (FMS)₃-exendin-4 Facilitates Prolonged Glucose Lowering Action in db/db Mice

The glucose lowering ability of exendin-4 and its (FMS)₃-exendin-4 derivative were compared in three groups of db/db mice (n=4 per group), following a single subcutaneous administration of a dose of 10 μg/mouse, and is summarized in FIG. 5. To avoid the daily variation in glucose concentrations, a characteristic of this diabetic rodent (Young et al., 1999), food was removed during the day, and glucose levels were monitored at the same time points in all three experimental groups. Results are expressed in FIG. 5 as percent decrease in blood glucose, relative to that of the PBS-treated group, monitored at the same time points during the day. Following subcutaneous administration of the native exendin-4, blood glucose level of the db/db mice fell by 48±3% two hours after administration, then increased with a t_{1/2} value of 15±2 hrs. With subcutaneously administered (FMS)₃-exendin-4, glucose levels fell by 50±3% two hours after administration, and remained at this low level over a period of 24 hrs before slowly returning to the levels of saline-injected db/db mice, with a t_{1/2} value of 31±2 hrs.

### REFERENCES

Eng J., Keinman W. A., Singh L., Singh G., Raufman J. P. (1992), “Isolation and characterization of exendin-4, an exendin-3 analogue, from Heloderma suspectum venom.” J. Biol. Chem. 267:7402-7405.

Gershonov E., Goldwaser I., Fridkin M., Shechter Y. (2000) “A novel approach for a water-soluble long-acting insulin prodrug: Design, preparation, and analysis of [(2-sulfo)-9-fluorenylmethoxycarbonyl]₃-insulin.” J. Med. Chem. 43:2530-2537.

Gershonov E., Shechter Y., Fridkin M. (1999) “New concept for long-acting insulin. Spontaneous conversion of an inactive modified insulin to the active hormone in circulation: 9-fluorenylmethoxycarbonyl derivative of insulin.” Diabetes 48:1437-1442.

Göke R., Fehmann H. C., Linn T., Schmidt H., Krause M. (1993) “Exendin-4 is a high potency agonist and truncated exendin-(9-39)-amide an antagonist at the glucagon-like peptide 1-(7-36)-amide receptor of insulin-secreting β-cells.” J. Biol. Chem. 268:19650-19655.

Gutnick M., Orskov C., Holst J. J., Ahren B., Efendic S. (1992) “Antidiabetogenic effect of glucagon-like peptide-1 (7-36) amide in normal subjects and patients with diabetes mellitus.” N. Engl. J. Med. 326:1316-1322.

Shechter Y., Precido-Patt L., Schreiber G., Fridkin M. (2001) “Prolonging the half-life of human interferon-α2 in circulation: Design, preparation, and analysis of (2-sulfo-9-fluorenylmethoxycarbonyl)₇-interferon-α2.” Proc. Natl. Acad. Sci. USA 98:1212-1217.

Young A. A. et al., (1999) “Glucose-lowering and insulin-sensitizing actions of exendin-4. Studies in obese diabetic (ob/ob, db/db) mice, diabetic fatty Zucker rats, and diabetic rhesus monkeys (Macaca mulatta).” Diabetes 48:1026-1034.

1. An exendin or exendin agonist derivative of the formula:
(X)ₙ-Z
wherein X is a radical 9-fluorenylmethoxycarbonyl (Fmoc) or 2-sulfo-9-fluorenylmethoxycarbonyl (FMS), Z is the residue of an exendin or exendin agonist linked to the radical X through an amino or hydroxyl group, and n is 1 to 3.

2. An exendin derivative of claim 1 wherein said exendin is exendin-4 [SEQ. ID. NO:1].

3. The exendin-4 derivative of claim 2 selected from the group consisting of the derivatives herein designated Fmoc-exendin-4, (Fmoc)₂-exendin-4, (Fmoc)₃-exendin-4, FMS-exendin-4, (FMS)₂-exendin-4 and (FMS)₃-exendin-4.

4. The exendin-4 derivative herein designated (FMS)₃-exendin-4.

5. An exendin derivative of claim 1 wherein said exendin is exendin-3 [SEQ. ID. NO:2].

6. The exendin-3 derivative of claim 5 selected from the group consisting of the derivatives herein designated Fmoc-exendin-3, (Fmoc)₂-exendin-3, (Fmoc)₃-exendin-3, FMS-exendin-3, (FMS)₂-exendin-3 and (FMS)₃-exendin-3.

7. An exendin agonist derivative of claim 1 wherein said exendin agonist is an exendin-3 or exendin-4 agonist.

8. An exendin agonist derivative of claim 7 wherein said exendin agonist is an exendin-4 agonist selected from the group of the sequences consisting of SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

9. A pharmaceutical composition comprising an exendin or exendin agonist derivative of claim 1, and a pharmaceutically acceptable carrier

10. A pharmaceutical composition comprising (FMS)₃-exendin-4 and a pharmaceutically acceptable carrier.

11. The pharmaceutical composition according to claim 9 for prevention of hyperglycemia.

12. The pharmaceutical composition according to claim 10 for prevention of hyperglycemia.

13. The pharmaceutical composition according to claim 9 for treatment of diabetes mellitus.

14. The pharmaceutical composition according to claim 13 for treatment of non-insulin dependent diabetes mellitus, insulin-dependent diabetes mellitus, or gestational diabetes mellitus.

15. The pharmaceutical composition according to claim 10 for treatment of diabetes mellitus.

16. The pharmaceutical composition according to claim 15 for treatment of non-insulin dependent diabetes mellitus, insulin-dependent diabetes mellitus, or gestational diabetes mellitus.

17. A method for prevention of hyperglycemia which comprises administering to an individual in need an effective insulinotropic amount of an exendin or exendin agonist derivative of claim 1.

18. A method for treatment of diabetes mellitus which comprises administering to an individual in need an effective amount of an exendin or exendin agonist derivative of claim 1.

19. The method of claim 18 wherein said diabetes mellitus is selected from the group consisting of non-insulin dependent diabetes mellitus, insulin-dependent diabetes mellitus, and gestational diabetes mellitus.


SEQUENCE LISTING (10 sequences)

SEQ ID NO: 1 - protein, 39 aa, Artificial
  Synthetic
HGEGTFTSDLSKQMEEEAVRLFIEWLKNGGPSSGAPPPS

SEQ ID NO: 2 - protein, 40 aa, Artificial
  Synthetic
HSDGTFITSDLSKQMEEEAVRLFIEWLKNGGPSSGAPPPS

SEQ ID NO: 3 - protein, 31 aa, Artificial
  Synthetic
HGEGTFTSDLSKQMEEEAVRLFIEWLKNGGP

SEQ ID NO: 4 - protein, 31 aa, Artificial
  Synthetic
HGEGTFTSDLSKQMEEEAVRLFIEWLKNGGY

SEQ ID NO: 5 - protein, 30 aa, Artificial
  Synthetic
HGEGTFTSDLSKQMEEEAVRLFIEWLKNGG

SEQ ID NO: 6 - protein, 30 aa, Artificial; identical to SEQ ID NO: 5
  Synthetic

SEQ ID NO: 7 - protein, 28 aa, Artificial
  Synthetic
HGEGTFTSDLSKQMEEEAVRLFIEWLKN

SEQ ID NO: 8 - protein, 39 aa, Artificial
  Synthetic
HGEGTFTSDLSKQLEEEAVRLFIEFLKNGGPSSGAPPPS

SEQ ID NO: 9 - protein, 28 aa, Artificial
  Synthetic
HGEGTFTSDLSKQLEEEAVRLFIEFLKN

SEQ ID NO: 10 - protein, 28 aa, Artificial
  Synthetic
HGEGTFTSDLSKQLEEEAVRLAIEFLKN